(19) United States
(12) Patent Application Publication
(10) Pub. No.: US 2017/0135559 A1
(43) Pub. Date: May 18, 2017

(54) DIRECT VISUALIZATION DEVICES, SYSTEMS, AND METHODS FOR TRANSSEPTAL CROSSING

(71) Applicants: BOSTON SCIENTIFIC SCIMED, INC., MAPLE GROVE, MN (US); MAYO FOUNDATION FOR MEDICAL EDUCATION AND RESEARCH, ROCHESTER, MN (US)

(72) Inventors: BENN HORRISBERGER, BLAINE, MN (US); DAVID JOHN LEHSE, OAKDALE, MN (US); JAMES K. CAWTHRA, JR., RAMSEY, MN (US); JAMES A. KLOS, BAY CITY, WI (US); JAMES P. ROHL, PRESCOTT, WI (US)

(73) Assignees: BOSTON SCIENTIFIC SCIMED, INC., MAPLE GROVE, MN (US); MAYO FOUNDATION FOR MEDICAL EDUCATION AND RESEARCH, ROCHESTER, MN (US)

(21) Appl. No.: 15/349,813

(22) Filed: Nov. 11, 2016

Related U.S. Application Data
(60) Provisional application No. 62/255,008, filed on Nov. 13, 2015.

(51) Int. Cl. A61B 1/00; A61B 1/018; A61B 1/04; A61B 1/07; A61B 1/313; A61B 1/005; A61B 1/06

(52) U.S. Cl. CPC A61B 1/00082; A61B 1/00165; A61B 1/00167; A61B 1/0051; A61B 1/018; A61B 1/0684; A61B 1/07; A61B 1/3137; A61B 1/042

(57) ABSTRACT

A direct visualization catheter adapted for transseptal crossing includes at least an outer member, and inner member, a transparent balloon member, and an imaging element. The outer member includes a tubular body extending from a proximal end to a distal end and defines a first lumen there through. The inner member is slidably disposed within the first lumen of the outer member and includes an elongate body with a distal end. The transparent balloon member is coupled between the distal end of the outer member and the distal end of the inner member such that the shape of the transparent balloon member is adjusted by sliding the inner member and the outer member relative to each other. The imaging element is disposed within the balloon member.

### CROSS-REFERENCE TO RELATED APPLICATIONS

This application claims the benefit of priority under 35 U.S.C. §119 to U.S. Provisional Application No. 62/255,008, filed Nov. 13, 2015, the entirety of which is incorporated herein by reference.

### TECHNICAL FIELD

The present invention relates to transseptal crossing devices, systems, and methods. For example, transseptal crossing devices provided herein include a direct visualization balloon with an adjustable balloon.

### BACKGROUND

Transseptal crossing is used to access the left atrium crossing from the right atrium through the septal wall. Prior to the use of transseptal crossing techniques, the left atrium was accessed via a transbronchial or direct percutaneous infrascapular approach. The left atrium can be accessed to assess hemodynamics and/or perform mitral valvuloplasty. The left atrium can also be accessed for atrial fibrillation (AF) ablation procedures. Typically a standard Brockenbrough needle is used to puncture the fossa ovalis during a transseptal crossing. The transseptal crossing of a catheter is typically guided using fluoroscopy and ultrasound. Transseptal crossings can also use echocardiography. Fluoroscopy is used to place the cathether and to confirm that the fassa ovalis has been tented, thus indicating that the correct location on the atrial septum has been identified. Fluoroscopy, however, cannot visualize soft tissue structures, thus ultrasound is typically used to confirm the trajectory of the crossing is appropriate so as not to pierce unintended structures. The use of fluoroscopy and ultrasound, however, still presents a risk of the transseptal crossing causing an aortic perforation, pericardial tamponade, systemic embolism, cerebral air embolism, or thrombus formation. Additionally, the use of fluoroscopy presents a risk to both the patient and medical personnel due to the prolonged exposure to radiation during a transseptal crossing procedure.

### SUMMARY

Disclosed herein are various embodiments of direct visualization devices, systems, and methods adapted for crossing the septum. Devises, systems, and methods provided herein include a direct visualization balloon having an adjustable shape and size to allow a medical technician or physician to have an optimized direct visualization in a blood field to conduct the transseptal crossing and an optimal shape for a minimally traumatic septal wall piercing.

In Example 1, a direct visualization catheter adapted for transseptal crossing includes an outer member, an inner member, a transparent balloon, and an imaging element. The outer member includes a tubular body extending from a proximal end to a distal end with the tubular body defining a first lumen there through. The inner member is slidably disposed within the first lumen of the outer member. The inner member includes an elongate body with a distal end. The transparent balloon member is coupled between the distal end of the outer member and the distal end of the inner member such that the shape of the transparent balloon member is adjusted by sliding the inner member and the outer member relative to each other. The imaging element is disposed within the balloon member.

In Example 2, a direct visualization catheter according to Example 1 has at least a portion of the transparent balloon member that defines a plurality of perforations adapted to allow inflation media to flow from within an interior cavity of the balloon member to an exterior surface of the balloon member.

In Example 3, a direct visualization catheter according to Example 2 is arranged such that the outer member defines a second lumen adapted to deliver an inflation media to the transparent balloon member.

In Example 4, a direct visualization catheter according to one of Examples 1-3 is arranged such that the imaging element is retained in a distal end of the outer member.

In Example 5, a direct visualization catheter according to one of Examples 1-4 further includes a light source disposed within an interior cavity of the balloon member and coupled to the distal end portion of the one or more elongate shafts. The light source can include a fiber optic bundle, single plastic optical fiber, an LED or some other illuminating device.

In Example 6, a direct visualization catheter according to one of Examples 1-5 is arranged such that the inner member defines a working lumen there through.

In Example 7, a direct visualization catheter according to one of Examples 1-6 where the transparent balloon member is a tubular sleeve having one end connected to the distal end of the outer member and the opposite end connected to the distal end of the inner member.

In Example 8, a direct visualization catheter according to one of Examples 1-7, where the distal end of outer tubular member has a tapered off-center profile.

In Example 9, a direct visualization catheter according to one of Examples 1-8, where the outer member further defines at least an illumination lumen adapted to retain an illuminating device.

In Example 10, a transseptal crossing system for accessing a left atrium from a right atrium of a heart includes the direct visualization catheter according to Example 6 and a piercing needle adapted to extend through the working channel to pierce the septal wall.

In Example 11, a transseptal crossing system according to Example 10 further includes at least one illumination device and the outer member defines at least one illumination lumen adapted to retain the at least one illumination device.

In Example 12, a transseptal crossing system according to Example 10 or Example 11 further includes a fastener or suturing device adapted to be delivered through the working channel.

In Example 13, a transseptal crossing system according to one of Examples 10-12 where at least a portion of the transparent balloon member defines a plurality of perforations adapted to allow inflation media to flow from within an interior cavity of the balloon member to an exterior surface of the balloon member and the outer member defines a second lumen adapted to deliver an inflation media to the transparent balloon member.

In Example 14, a transseptal crossing system according to one of Examples 10-13 where the distal end of the outer member has a tapered off-center profile and the imaging element retained in the distal end of the outer member along a tapered edge.

In Example 15, a transseptal crossing system according to one of Examples 10-14 where the transparent balloon member is a tubular sleeve having one end connected to the distal end of the outer member and the opposite end connected to the distal end of the inner member.

In Example 16, a method of accessing the left atrium includes delivering a direct visualization catheter into a right atrium, the direct visualization balloon including an outer member, an inner member, and a transparent balloon member, the outer member including a tubular body extending from a proximal end to a distal end, the tubular body defining a first lumen there through, the inner member being slidably disposed within the first lumen of the outer member, the inner member including an elongate body with a distal end, the transparent balloon member being coupled between the distal end of the outer member and the distal end of the inner member such that the shape of the transparent balloon member is adjusted by sliding the inner member and the outer member relative to each other; inflating the transparent balloon member in the right atrium with an inflation media; visualizing the septum wall in the right atrium using the direct visualization catheter while the inner member is in a retracted state relative to the outer member to identify a desired crossing location; deflating the transparent balloon member and extending the inner member relative to the outer member; and passing the direct visualization catheter through the septum and into the left atrium.

In Example 17, the method of Example 16 further includes piercing the septum by passing a piercing tool through a working channel in the inner member.

In Example 18, the method of Example 16 or Example 17 further includes retracting the inner member relative to the outer member when the distal ends are in the left atrium and inflating the transparent balloon member in the left atrium to visualize the left atrium.

In Example 19, the method of one of Examples 16-18 further includes conducting an ablation procedure in the left atrium.

In Example 20, the method of Example 19 includes delivering an ablation tool through a channel through the inner member and placing it on damaged tissue. The ablation tool being adapted to use radio frequency or laser methods to ablate the damaged tissue.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1A is an illustration of a direct visualization catheter in a retracted and inflated condition.

FIG. 1B is an illustration of a direct visualization catheter in an extended and deflated condition.

FIGS. 2A-2C illustrate an example of how a direct visualization catheter provided herein can be used to access the left atrium.

FIGS. 3A and 3B depict side views of an example of a direct visualization catheter in an extended and deflated condition.

FIGS. 4A and 4B are illustrations of an atraumatic tip that can be used in direct visualization catheters provided herein.

While the devices and system provided herein are amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Direct visualization devices, systems and methods provided herein can improve the safety of accessing the left atrium from the right atrium. Direct visualization devices, systems and methods provided herein can include features that allow for a direct visualization balloon to pass through small apertures without damaging the surrounding tissue or the direct visualization balloon, thus further minimizing the invasiveness of accessing the left atrium. Direct visualization devices, systems, and methods provided herein can allow for the shape of the direct visualization balloon to be modified to optimize the visualization of surrounding tissues. In some cases, direct visualization devices, systems, and methods provided herein can be used to deliver an artificial heart valve, to surgically repair a heart valve, to provide an AF ablation therapy, or to deliver a therapeutic agent or diagnostic device to select portions of the left atrium. Exemplary procedures include those that bicuspidizes a tricuspid valve, edge to edge stitching techniques (or Alfieri stitches), mitral valve stitches, closures of paravalvular leaks, percutaneous paravalvular leak closure, and/or percutaneous closure of prevalvular leaks. The term “suture” is used herein to refer to any fastening of anatomical structures, which can be made with any suitable fastener including suturing thread, clips, staples, hooks, tacks, clamps, etc. Direct visualization devices, systems, and methods provided herein can also be used to visualize anatomical structures other than the atria of the heart and or to deliver suitable therapies. In some cases, systems, devices, and methods provided herein can suture one or more heart valve leaflets.

Direct visualization devices, systems, and methods provided herein can allow for balloon catheter visualization of a target location, which can provide anatomy and pathology identification as well as device placement visual feedback to the physician user during a minimally invasive method. Direct visualization devices, systems, and methods provided herein can include an elongate, compliant balloon having a transparent wall. In some case, the balloon can include apertures (e.g., pores) to allow for the balloon to “weep” to provide a visually clear area surrounding the balloon. In some cases, the balloon wall (e.g., a transparent balloon wall) can include a silicone material. In some cases, transparency of the devices described herein or portions thereof are suitable for visibility in the visible range, e.g., radiation wavelengths ranging from about 390 nanometers (nm) to about 700 nm. In some cases, the transparency of the devices described herein can allow for visibility suitable for monochromatic imaging and/or imaging in non-visible ranges (e.g., IR).

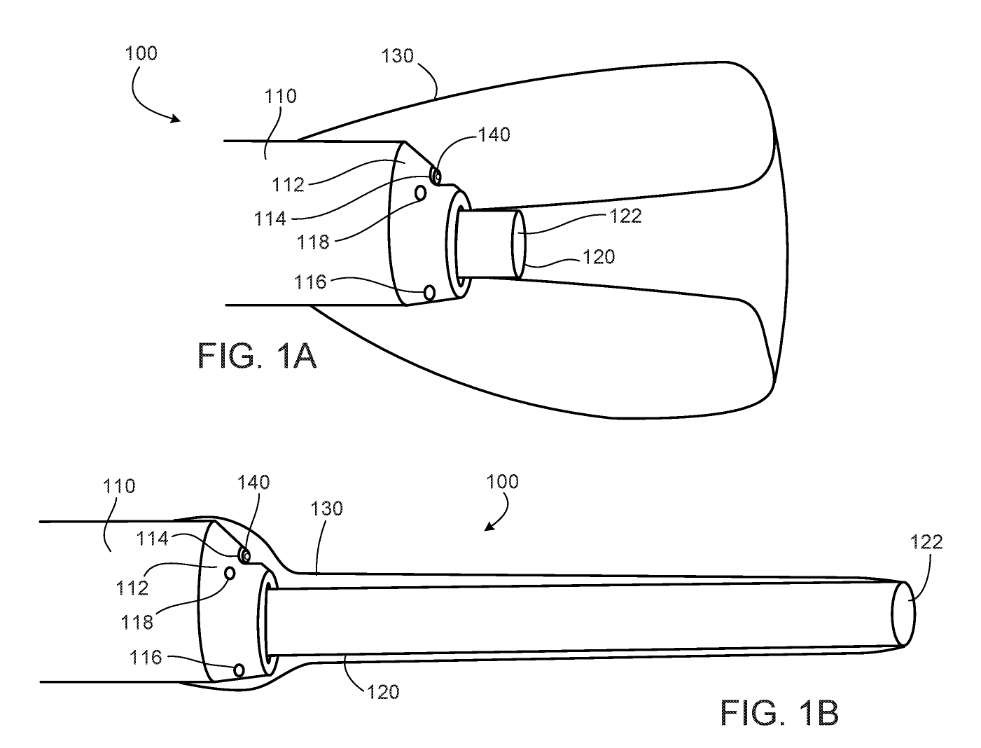

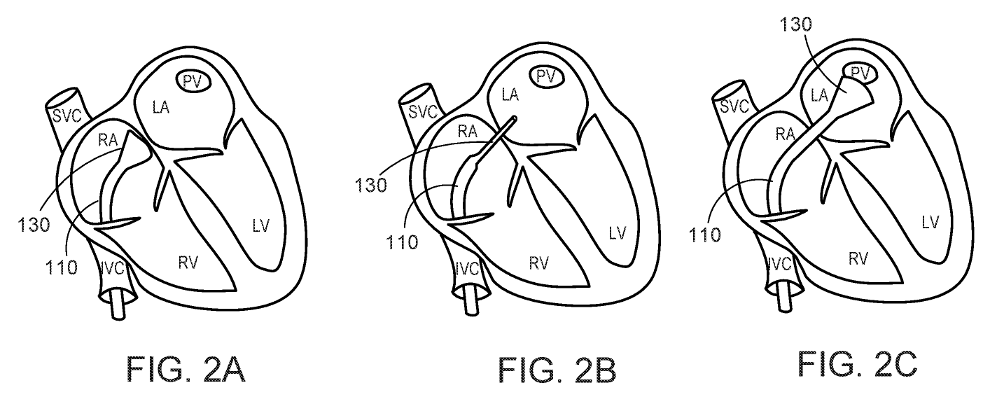

FIG. 1A illustrates a distal end of an exemplary direct visualization catheter 100 in a retracted position with a partially inflated direct visualization balloon. FIG. 1B illustrates the distal end of catheter 100 in an elongated position and having a deflated balloon. As shown, the elongated position of FIG. 1B can minimize the profile of the direct visualization catheter. FIGS. 2A-2C depict how the direct visualization catheter 100 can be used to access the left atrium from the right atrium.

As shown in FIGS. 1A and 1B, catheter 100 includes an outer member 110, and inner member 120, a balloon 130, and an imaging element 140. Catheter 100 can be a steerable catheter The inner member 120 resides in a first lumen defined by the outer member such that the inner member and outer member can slide relative to each other between at least a retracted position (e.g., FIG. 1A) and an extended position (e.g., FIG. 1B). Balloon 100 can be a sleeve having one end sealed to a distal end of outer member 110 and an opposite end sealed to a distal end of inner member 120 such that balloon 130 forms a donut shaped direct visualization balloon when the members are in the retracted position and the balloon is inflated with an inflation medium. When in an extended position, balloon 130 can form a single layer over the sides of the inner member and the outer member, as shown in FIG. 1B. Because the extended position allows for the balloon to not overlap along the sizes of catheter 100, the profile of catheter 100 is reduced as compared to catheters that allow a deflated balloon to form multiple layers along the sides of the catheter.

Referring still to FIGS. 1A and 1B, outer member 110 can include a tapered tip 112. Tapered tip 112 can reduce the trauma associated with the outer member passing through a narrow body passageway or aperture and also reduce the probability of balloon 130 ripping as catheter 100 is inserted into a left atrium. As shown, tapered tip 112 is off-centered. In some cases, tapered tip 112 can be stub nosed. As discussed in more detail below, tapered tip 112 can be arranged to minimize visual obstructions. Outer member 110 also includes additional lumens 114, 116, and 118, all having a distal aperture in tapered tip 112. Lumen 114 can provide a passageway for imaging element 140 to provide visual image data to a proximal end of direct visualization catheter 100 such that the physician can view tissues surrounding balloon 130. Lumen 118 (or multiple lumens) can provide a passageway for light source (e.g., plastic optical fibers or other optical fibers) to provide light for to balloon 130. Lumen 116 can provide a passageway for an inflation medium to inflate balloon 130. Lumen 116 can also be used to suck inflation media out of balloon 130 to deflate balloon 130. In some cases, a single lumen can be used for a combination of providing light, inflation media, and/or imaging. In some cases, inflation media can be provided to balloon 130 by delivering the inflation media in the first lumen around inner member 120. Inner member 120 can also define a central working cannel 122, which can be used to pass devices, therapeutics, or tools into a working space (e.g., in the left atrium).

Imaging element 140 can be any suitable device that provides images of tissues surrounding balloon 130. The imaging element 140 can be used to obtain images of tissue in a blood-field environment, for example, within the heart or a blood vessel. In some cases, the imaging element 140 can include, but is not limited to, optical elements (e.g., lens), a sensor, or a combination thereof, for capturing an image within a patient's anatomy. In some cases, a portion of the imaging element 140 may be disposed within the balloon member. In some cases, a portion of the imaging element 140 may be disposed within a shaft portion, a manifold, or a location external to the devices described herein, for example, a wireless imaging sensor, or other imaging component. For instance, in some cases, the imaging element 140 can include at least one component (e.g., lens) that is arranged within the balloon while another component (e.g., a sensor) is disposed on a different area of the device, or separate from and within proximity of the device.

In some cases, imaging element 140 can be an integrated camera or an integrated solid-state-camera system, such as a charge-coupled device (CCD) or complementary metal—oxide—semiconductor (CMOS) imaging system, for visualizing tissue. In some cases, the imaging element 140 can include an ultrasound sensor or device. In some cases, imaging element 140 can include a fiber optic based device.

Referring to FIG. 2A, catheter 100 can be inserted into a right atrium RA of a heart through the femoral vein to the superior vena cava or the interior vena cava, or any other suitable artery or vein. During insertion through an artery or vein, inner member 120 is in an extended position relative to outer tube 110 to minimize the profile of catheter 100. Cather 100 can be a steerable catheter using any suitable technique for guiding the movement of the distal tip of catheter 100 through arteries or veins to the right atrium RA. As shown in FIG. 2A, prior to crossing the septum into the left atrium LA, balloon 130 can be inflated to visually inspect tissues in the right atrium RA to ensure than an appropriate crossing location is chosen. Balloon 130 can be inflated by shifting the relative positions of inner member 120 to outer member 110 to a retracted position, as shown in FIG. 1A. Any appropriate technique can be used to visually identify an appropriate crossing location. For example, a physician can navigate the vasculature and locate the right atrium, septal wall, limbus and fassa ovalis. After a physician or medical technician has visually confirmed working channel 122 to be in a correct location for crossing the septum, balloon 130 can be deflated and inner member 120 extended such that catheter 100 takes on the shape of a small dilator having a minimized profile.

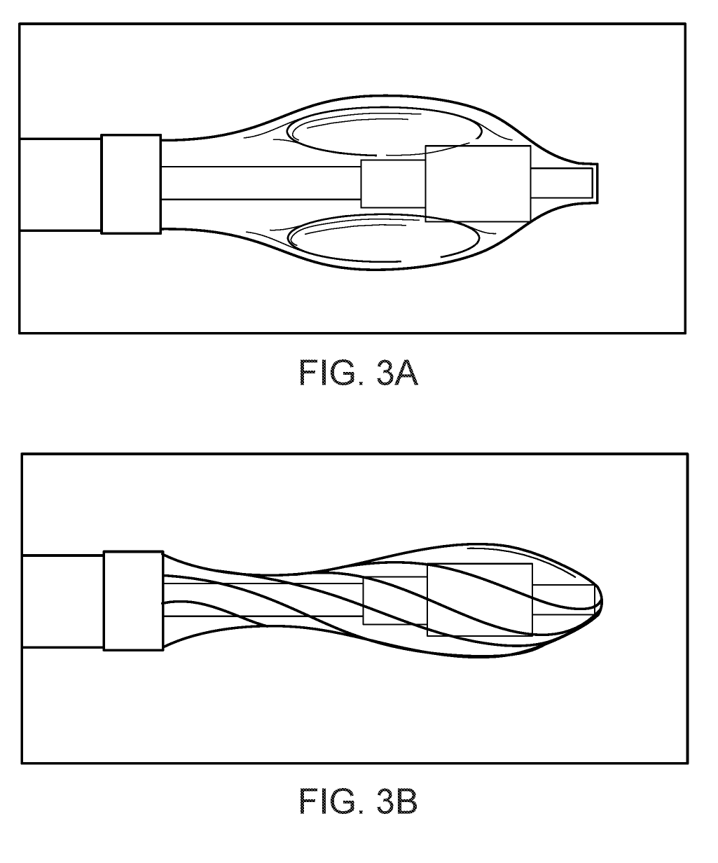

Once extended (such as in FIG. 1B), catheter 100 can be advanced forward and pass across the septum into the left atrium, such as shown in FIG. 2B. Because of the minimized profile, catheter 100 can pass through the septum with minimal trauma to the septum. When in the extended position, balloon 130 can sometimes form ripples extending along the length of the catheter, such as shown in FIG. 3A. These ripples, however, can easily deform as catheter 100 is passed through the septum. In some cases, inner member 120 can be twisted relative to outer member 110 such that the ripples spiral as shown in FIG. 3B in order to further reduce the profile of catheter 100 in an extended position.

In some cases, prior to passing across the septum, the septum can be pierced by passing a piercing tool through working channel 122. In some cases, a piercing tool (e.g., a needle, a guide wire) can pierce the septum while balloon 130 is inflated so that a physician or medical technician can visualize the piercing operation. After catheter 100 is in left atrium LA, inner member 120 can be retracted and balloon 130 inflated to provide direct visualization of the left atrium, such as shown in FIG. 2C. Once in left atrium LA, catheter 100 can be used to deliver any suitable device, treatment, or therapeutic to the left atrium. In some cases, surgical tools can be passed through working channel 122 to surgically repair a heart valve. In some cases, catheter 100 can be adapted to provide an AF ablation therapy. For example, an ablation device could be passed through the working channel and accurately placed on damaged tissue. In some cases the ablation tool might be use radio frequency or laser methods for ablating. In some cases, electrophysiology mapping catheters and ablation catheters are passed through an appropriately sized working channel in catheter 100 to gain access to the left atrium for mapping and ablation procedures.

Any suitable inflation medium can be used to inflate balloon 130. In some case, the inflation media includes saline. As discussed above, lumen 116 can be used to deliver the inflation media. In some cases, multiple lumens can be adapted to jet inflation media, e.g., saline, into balloon 130. A manifold can connect an external fluid supply to one or more lumens of outer member 110. In some cases, a flexible tubing, sometimes referred to as a strain relief tubing, is coupled between the manifold and lumen 116 of outer member 110 at the proximal end of the catheter 100. Flexible tubing can help to increase kink resistance of catheter 100.

In some cases, balloon 130 can include tear lines that define pledgets having tear lines, or weakened sections, in the balloon wall that define pledgets adapted to be sutured to anatomical locations and separated from balloon 130.

Each lumen in outer member 110 and inner member 120 can be formed from one of various cross-sectional shapes, e.g., circle, oval, slot, square, rectangular, triangular, trapezoid, rhomboid, or irregular shape. The shape of the lumen may facilitate receiving other components of the imaging element 140, an illuminating element (e.g., fiber optic light cables), or inner member 120.

Balloon 130 of catheter 100 can be a weeping balloon. Weeping balloon, in the context of the present disclosure, includes a balloon structure defining one or more perforations (also described as apertures or micropores, extending through a balloon wall). As such, weeping balloons can transfer inflation media through the balloon wall, from interior cavity to exterior surface of balloon 1340. Transferring inflation media to exterior surface can provide a benefit of displacing blood from exterior surface of balloon 130 that would otherwise blur or obstruct visual imaging through balloon 130. In other words, inflation media transferred through the one or more perforations can help keep the exterior surface visually clear. If you just put a balloon against an anatomical surface, blood can be trapped on the balloon surface and thus obscures the view, but inflation media (e.g., saline) exiting the pores of a weeping balloon can wash away this blood on the balloon surface adjacent to the wall. In some cases, a weeping balloon used in a balloon catheter visualization system or device provided herein can have at least 3 punctured holes. In some cases, weeping balloons used in direct visualization systems or devices provided herein can have between 3 and 10,000 puncture holes, between 3 and 1,000 puncture holes, between 3 and 100 puncture holes, or between 3 and 10 puncture holes. In some cases, the number and dimensions of puncture holes in a weeping balloon used in a balloon catheter visualization system or device provided herein allows for an inflation media flow rate of between 1 and 50 ml/minute. In some cases, systems and methods provided herein control an inflation media flow rate to be between 3 ml/minute and 10 ml/minute. In some cases, a weeping balloon used in direct visualization systems and devices provided herein can have hundreds of holes that perfuse inflation media (e.g., saline) through the balloon and into the blood. In some cases, a weeping balloon used in a balloon catheter visualization system or device provided herein can have a greater pore density in portions of the balloon wall in the center of the field of view and a lower pore density around a periphery of the field of view.

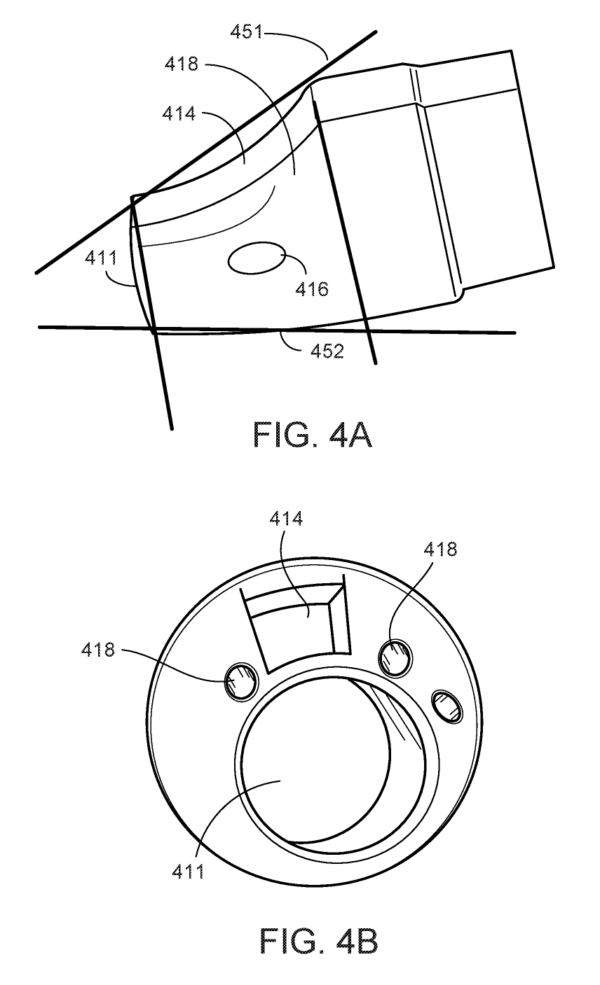

A distal end of outer member 110 has a tapered tip 112. FIGS. 4A and 4B depict an embodiment of an atraumatic tapered tip that can be used as tapered tip 112 in catheter 100. FIG. 4A is a side view of the atraumatic tip. FIG. 4B is a front view of the atraumatic tip. As shown, the atraumatic tip includes a first lumen 411 for receiving the inner member, such as inner member 120 depicted in FIGS. 1A and 1B. Atraumatic tip additionally includes lumen 414 for holding an imaging element, lumen 416 for delivering inflation media, and lumens 418 for providing light. For example, lumen 414 can hold a digital camera and lumens 418 can retain plastic optical fibers for delivering light to a direct visualization balloon. In addition to allowing the passage of inflation fluid (e.g., saline) into a direct visualization balloon, lumen 416 can additionally allow for the passage of surgical tools into the direct visualization balloon. The shape of the atraumatic tip is such that it has an atraumatic taper 451 on the side of the tip having the imaging element lumen 414 and a non-occluding stub nose 452 along an opposite side of the atraumatic tip. The angle of the atraumatic taper 451 is such that it does not occlude too much of the image captured by the imaging element. Additionally, the taper around each side can allow the entire device to pierce and then pass through the septal wall with minimal trauma.

A number of embodiments of the direct visualization devices, systems, and methods have been described. Nevertheless, it will be understood that various modifications may be made without departing from the spirit and scope of the subject matter described herein. For example, lighting may be provided by either a fiber optic bundle, a single plastic optical fiber, an LED or some other illuminating device. Accordingly, other embodiments are within the scope of the following claims.

What is claimed is:

1. A direct visualization catheter adapted for transseptal crossing comprising:
an outer member comprising a tubular body extending from a proximal end to a distal end, the tubular body defining a first lumen there through;
an inner member slidably disposed within the first lumen of the outer member, the inner member comprising an elongate body with a distal end;
a transparent balloon member coupled between the distal end of the outer member and the distal end of the inner member such that the shape of the transparent balloon member is adjusted by sliding the inner member and the outer member relative to each other; and
an imaging element disposed within the balloon member.

2. The direct visualization catheter of claim 1, wherein at least a portion of the transparent balloon member defines a plurality of perforations adapted to allow inflation media to flow from within an interior cavity of the balloon member to an exterior surface of the balloon member.

3. The direct visualization catheter of claim 2, wherein the outer member defines a second lumen adapted to deliver an inflation media to the transparent balloon member.

4. The direct visualization catheter of claim 1, wherein the imaging element retained in a distal end of the outer member.

5. The direct visualization catheter of claim 1, further comprising a fiber optics light source disposed within an interior cavity of the balloon member and coupled to the distal end portion of the one or more elongate shafts.

6. The direct visualization catheter of claim 1, wherein the inner member defines a working lumen there through.

7. The direct visualization catheter of claim 1, wherein the transparent balloon member is a tubular sleeve having one end connected to the distal end of the outer member and the opposite end connected to the distal end of the inner member.

8. The direct visualization catheter of claim 1, wherein the distal end of outer tubular member has a tapered off-center profile.

9. The direct visualization catheter of claim 1, wherein the outer member further defines at least an illumination lumen adapted to retain an illuminating device.

10. A transseptal crossing system for accessing a left atrium from a right atrium of a heart comprising:
the direct visualization catheter of claim 16; and
a piercing needle adapted to extend through the working channel to pierce the septal wall.

11. The system of claim 10, further comprising at least one illumination device, wherein the outer member defines at least one illumination lumen adapted to retain the at least one illumination device.

12. The system of claim 10, further comprising a fastener or suturing device adapted to be delivered through the working channel.

13. The system of claim 10, wherein at least a portion of the transparent balloon member defines a plurality of perforations adapted to allow inflation media to flow from within an interior cavity of the balloon member to an exterior surface of the balloon member, wherein the outer member defines a second lumen adapted to deliver an inflation media to the transparent balloon member.

14. The system of claim 10, wherein the distal end of the outer member has a tapered off-center profile and the imaging element retained in the distal end of the outer member along a tapered edge.

15. The system of claim 10, wherein the transparent balloon member is a tubular sleeve having one end connected to the distal end of the outer member and the opposite end connected to the distal end of the inner member.

16. A method of accessing the left atrium comprising:
delivering a direct visualization catheter into a right atrium, the direct visualization balloon comprising an outer member, an inner member, and a transparent balloon member, the outer member comprising a tubular body extending from a proximal end to a distal end, the tubular body defining a first lumen there through, the inner member being slidably disposed within the first lumen of the outer member, the inner member comprising an elongate body with a distal end, the transparent balloon member being coupled between the distal end of the outer member and the distal end of the inner member such that the shape of the transparent balloon member is adjusted by sliding the inner member and the outer member relative to each other;
inflating the transparent balloon member in the right atrium with an inflation media;
visualizing the septum wall in the right atrium using the direct visualization catheter while the inner member is in a retracted state relative to the outer member to identify a desired crossing location;
deflating the transparent balloon member and extending the inner member relative to the outer member; and
passing the direct visualization catheter through the septum and into the left atrium.

17. The method of claim 16, further comprising piercing the septum by passing a piercing tool through a working channel in the inner member.

18. The method of claim 16, further comprising retracting the inner member relative to the outer member when the distal ends are in the left atrium and inflating the transparent balloon member in the left atrium to visualize the left atrium.

19. The method of claim 16, further comprising conducting an ablation procedure in the left atrium.

20. The method of claim 19, wherein an ablation tool is delivered through a channel through the inner member and placed on damaged tissue, the ablation tool being adapted to use radio frequency or laser methods to ablate the damaged tissue.
